(19) United States
(12) Patent Application Publication
(10) Pub. No.: US 2008/0286815 A1
(43) Pub. Date: Nov. 20, 2008

(54) SPECTROPHOTOMETRIC ASSAY FOR HUMAN HISTONE DEACETYLASE 8

(75) Inventors: Weiping ZHENG, Copley, OH (US); David George FATKINS, Clinton, OH (US)

Correspondence Address: ROETZEL AND ANDRESS, 222 SOUTH MAIN STREET, AKRON, OH, 44308, US

(73) Assignee: THE UNIVERSITY OF AKRON, Akron, OH (US)

(21) Appl. No.: 12/117,951

(22) Filed: May 9, 2008

Related U.S. Application Data
(60) Provisional application No. 60/928,704, filed on May 11, 2007.

(51) Int. Cl. G01N 33/567; C12Q 1/34

(52) U.S. Cl. 435/7.23; 435/18

(57) ABSTRACT

The present invention relates to the development of a novel assay for determining the activity of human histone deactylase 8 (HDAC8). As shown in FIG. 3, thioactyl-lysine-containing peptide is used as the substrate for the HDAC8-catalyzed dethioacetylation reaction. Thioacetate, which is formed during this reaction, is subsequently reacted with Ellman's reagent, 5,5′-dithiobis(2-nitrobenzoate), (DTNB) with a quantitative formation of 2-nitro-5-thiobenzoate (TNB). The concentration of thioacetate formed during the conversion reaction can be quantified by measuring the absorbance of TNB at 412 nm.

The invention relates to a spectrophotometric assay for determining the activity of human histone deacetylase 8 (HDAC8). More specifically, the invention relates to a spectrophotometric assay that is selective for HDAC8 as compared to other human histone deacetylases.

### BACKGROUND OF THE INVENTION

Protein acetyltransferases and protein deacetylases are the two families of enzymes that respectively catalyze the specific lysine N^{ε}-acetylation and N^{ε}-deacetylation on proteins such as the core histone proteins, various transcription factors, alpha-tubulin, acetyl-coenzyme A synthetases and human immunodeficiency virus (HIV) Tat protein that are, respectively, involved in gene transcriptional, cytoskeletal, metabolic control and HIV infection. (See FIG. 1) Acetyltransferase-catalyzed creation, deacetylase-catalyzed destruction, and bromodomain-mediated specific recognition of N^{ε}-acetyl-lysine on proteins have been regarded as an emerging intracellular signaling mechanism reminiscent of the signaling mechanism defined by protein posttranslational phosphorylation and dephosphorylation on serine, threonine, and tyrosine side chain OH groups.

Based on homology with yeast transcriptional repressors, phylogenetic analysis, and different cofactor requirements, human protein deacetylase enzymes have been categorized into class I, II, III, and IV subfamilies. Class I includes HDAC1, 2, 3 and 8. Class II includes HDAC4, 5, 6, 7, 9, and 10. Class III includes SIRT1, 2, 3, 4, 5, 6, and 7. Class IV includes HDAC11 and its related enzymes. Class I, II, and IV enzymes, also collectively known as classical enzymes, all require a catalytic zinc (Zn²⁺) for activity, whereas the class III enzymes, also known as sirtuins, require coenzyme nicotinamide adenine dinucleotide (NAD⁺) for activity.

The classical protein deacetylase-catalyzed reaction has been targeted for developing novel therapies for cancer. In fact, suberoylanilide hydroxamic acid (SAHA) has recently been approved as the first classical protein deacetylase inhibitor by US Food and Drug Administration for treating cutaneous T cell lymphoma (CTCL). Several other small molecule inhibitors for the classical enzymes have also moved onto different stages of clinical trials to primarily evaluate their anti-cancer profiles. These developments have fueled ongoing efforts in both industry and academia to pursue further inhibitors, especially the isoform selective inhibitors for classical protein deacetylase enzymes.

One prominent example is the demonstration that HDAC8-selective inhibition resulted in cell death in T-cell lymphomas (Balasubramanian et al, Leukemia, 2008 Feb. 7. [Epub ahead of print]). One important requirement for having an efficient overall process of developing inhibitors for these human enzymes as novel drugs is to have an efficient assay platform for the quantitative measurement of the activity of these human enzymes. In terms of determining the HDAC8 activity, radioactive assay, HPLC-based assay, and fluorescence assay are the three currently existing assay formats (Riester et al, Anal. Biochem., 362 (2007) 136-141; Heltweg et al, Methods, 36 (2005) 332-337). However, these assays suffer from the following drawbacks: not user-friendly in terms of the necessity to work with radioactive materials, slow in terms of using HPLC to achieve quantitative measurements, tedious, expensive, and not HDAC8-selective. Therefore, a need exists to develop more convenient and cost-effective assay platforms for a selective, quantitative measurement of HDAC8 activity.

The present invention is directed toward developing such an assay platform to quantitatively measure the activity of HDAC8. The present invention is based on the previous observations made by the inventors of the present invention. The inventors of the present invention previously synthesized two human p53 tumor suppressor protein C-terminal peptides (amino acid residue 372-389) containing N^{ε}-thioacetyl-lysine (ThAcK) or N^{ε}-acetyl-lysine at the 382 position. FIG. 2 shows a structural comparison of N^{ε}-thioacetyl-lysine and N^{ε}-acetyl-lysine. When these two peptides were incubated with HDAC8, the inventors of the present invention have determined that HDAC8 could catalyze the removal of the thioacetyl group and the acetyl group with comparable rates (D. G. Fatkins, A. D. Monnot, W. Zheng, Bioorg. Med. Chem. Lett., 16 (2006) 3651-3656). Therefore, these two peptides are determined to be functional mimics of each other for purposes of this invention. As used herein, the term “functional mimic” and variations thereof are used as defined in the foregoing.

### SUMMARY OF THE INVENTION

The invention relates to a spectrophotometric assay for determining the activity of human histone deacetylase 8 (HDAC8). More specifically, the invention relates to a spectrophotometric assay that is selective for HDAC8 as compared to other human histone deacetylases.

In one embodiment, the present invention provides a process to quantitatively measure the activity of human histone deactylase 8 (HDAC8) enzyme, the process comprising: a) providing a peptide substrate including at least one ThAcK amino acid; b) providing HDAC8; c) allowing the HDAC8 to react with the peptide substrate to produce at least one peptide product and thioacetate; d) quenching HDAC8 activity; e) adding DTNB to the quenched HDAC8; f) allowing DTNB to react with thioacetate to produce TNB; and g) quantitatively measuring the activity of HDAC8 by measuring the thioacetate concentration from the absorbance of TNB at 412 nm.

In another embodiment, the present invention provides a selective peptide substrate comprising at least one ThAcK amino acid, wherein the at least one ThAcK amino acid has the formula

R¹NH—φₘ—(ThAcK)—φₙ—COR²

wherein R¹ is selected from the group consisting of H, acetyl (CH₃CO), and terbutyloxycarbonyl (^{t}Boc); R² is selected from the group consisting of hydroxyl (OH), amino (NH₂), and 7-amino-4-methylcoumarine (AMC); φ is a naturally occurring L-amino acid; ThAcK is L-N^{ε}-thioacetyl-lysine; m is 0-10; and n is 0-7 and when m and/or n is >2, φ may be the same amino acid or may be different amino acids.

In yet another embodiment, the present invention provides a selective peptide substrate comprising at least one ThAcK amino acid and the use thereof as a reagent for selectively reporting the activity of HDAC8 inside human cells.

In still another embodiment, the present invention provides a method for the in vivo or the in vitro screening for cancer-treating drugs by a) providing human cancer cells treated with a cancer-treating drug, wherein the cancer cells contain a mixture of human enzymes, one being HDAC8; b) lysing the cells; c) providing a peptide substrate including at least one ThAcK amino acid having the general formula

R¹NH—φₘ—(ThAcK)—φₙCOR²

wherein R¹ is selected from the group consisting of H, acetyl (CH₃CO), and terbutyloxycarbonyl (^{t}Boc); R² is selected from the group consisting of hydroxyl (OH), amino (NH₂), and 7-amino-4-methylcoumarine (AMC); φ is a naturally occurring L-amino acid; ThAcK is L-N^{ε}-thioacetyl-lysine; m is 0-10; and n is 0-7, and n is 0-7 and when m and/or n is >1, φ may be the same amino acid or may be different amino acids; d) contacting the peptide substrate with the cancer cell lysates; e) allowing the HDAC8 in the cancer cell lysates to react with the peptide substrate to produce at least one peptide product and thioacetate; and f) quantitatively measuring the thioacetate concentration of the reaction of step (e) to determine the activity of HDAC8.

These and other embodiments of the present invention will be appreciated by the one skilled in the art upon reading and understanding of the disclosure and teaching provided herein.

### BRIEF DESCRIPTION OF THE FIGURES

FIG. 1 is an illustration of the lysine N^{ε}-acetylation and deacetylation reactions catalyzed respectively by protein acetyltransferases and protein deacetylases.

FIG. 2 is a structural comparison of N^{ε}-acetyl-lysine and N^{ε}-thioacetyl-lysine according to the invention.

FIG. 3 the reaction scheme for the DTNB-based spectrophotometric assay, according to the invention.

FIG. 4 provides a comparative analysis, based on HPLC-based assay data and UV-Vis DTNB-based spectrophotometric assay data of enzymatic dethioacetylation of the thioacetyl-lysine peptide to form peptide (product), showing the quantification of the released p53 peptide.

FIG. 5 is the structure of the L-N^{α}-Fmoc-N^{ε}-thioacetyl-lysine.

### DETAILED DESCRIPTION OF THE INVENTION

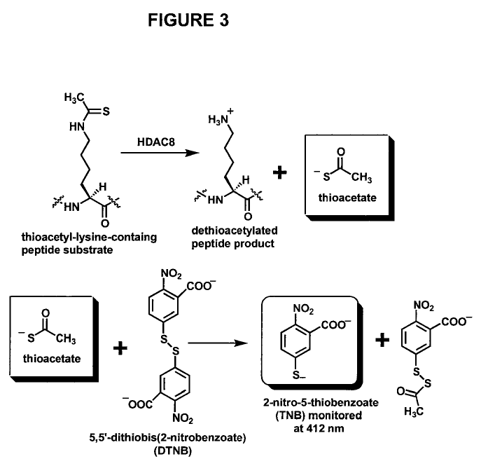

The present invention relates to the development of a novel assay for determining the activity of human histone deactylase 8 (HDAC8). As shown in FIG. 3, a thioacetyl-lysine-containing peptide is used as the substrate for the HDAC8-catalyzed dethioacetylation reaction. Thioacetate, which is formed during this reaction, is subsequently reacted with Ellman's reagent, 5,5′-dithiobis(2-nitrobenzoate) (DTNB) with a quantitative formation of 2-nitro-5-thiobenzoate (TNB). The concentration of thioacetate formed during the conversion reaction can be quantified by measuring the absorbance of TNB at 412 nm.

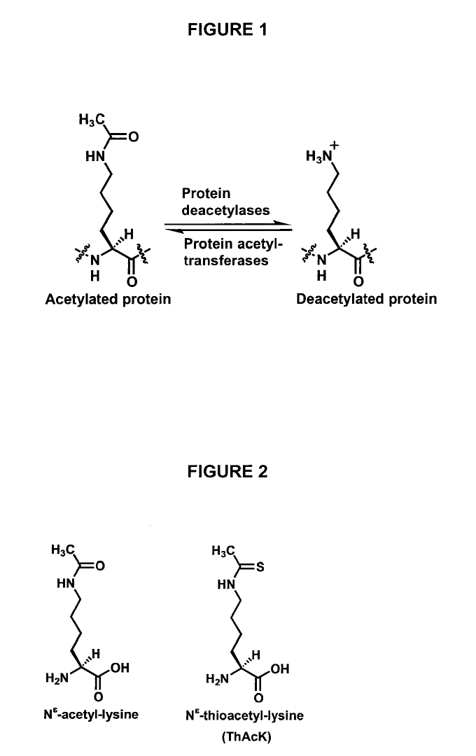

The inventors have shown that the thioacetyl group can serve as a functional mimic for the acetyl group for enzymatic deacetylation reactions catalyzed by HDAC8. In one embodiment of the invention, therefore, the inventors, based on their identification of the structural mimicry shown in FIG. 2, provide a spectrophotometric HDAC8 assay. With further reference to FIG. 3, this reaction scheme is for the DTNB-based spectrophotometric assay according to the present invention. The peptide sequence, or substrate, that harbors the novel building block (ThAck) according to the invention, has the general formula

R¹NH —φₘ—(ThAcK)—φₙ—COR²

wherein R¹ is selected from the group consisting of H, acetyl (CH₃CO), and terbutyloxycarbonyl (^{t}Boc); R² is selected from the group consisting of hydroxyl (OH), amino (NH₂), and 7-amino-4-methylcoumarine (AMC); φ is a naturally occurring L-amino acid; ThAcK is L-N^{ε}-thioacetyl-lysine; m is 0-10; and n is 0-7 and when m and/or n is >2, φ may be the same amino acid or may be different amino acids. This peptide sequence or substrate is first allowed to incubate with the enzyme HDAC8, as shown in the reaction scheme. The incubation may be conducted at room temperature, or another appropriate temperature as known to those skilled in the art. (See D. G. Fatkins, et al., Bioorg. Med. Chem. Lett. 16 (2006) 3651-3656, incorporated herein in its entirety by reference.) The peptide substrate, in the presence of HDAC8, is converted to a peptide product and thioacetate. The reaction is then quenched, stopping the conversion process, and the Ellman's reagent, 5,5′-dithiobis(2-nitrobenzoate) (DTNB), is added and selectively reacts with the thioacetate to produce 2-nitro-5-thiobenzoate (TNB). As is stated above, by measuring the absorbance of TNB at 412 nm, the concentration of thioacetate formed during the conversion reaction can be quantified.

The following abbreviations, as used herein, refer to: HDAC is histone deacetylase; NAD⁺ is nicotinamide adenine dinucleotide; SAHA is suberoylanilide hydroxamic acid; CTCL is cutaneous T cell lymphoma; DTNB is 5,5′-dithiobis(2-nitrobenzoic acid); TNB is 2-nitro-5-thiobenzoate; and HPLC is high-pressure liquid chromatography.

Further, the term “quantitative measurement” refers to the determination of the amount and the concentration of a substance, such as a chemical, in this case produced by the identified reaction.

In addition, the term “activity” as used herein means the amount of a product formed in a given period of time with a given amount of the enzyme that catalyzes the formation of the product from a given amount of substrate.

DTNB has been known to be able to selectively react with thiol-containing substances including free thiols (e.g. free cysteine or the cysteine residues on a denatured protein molecule) and thioacids (e.g. thioacetic acid, thiobenzoic acid, or a peptidic thioacid), with the released TNB having maximum absorbance at 412 nm. (See P. W. Riddles, R. L. Blakeley, B. Zerner, Reassessment of Ellman's Reagent, Methods Enzymol. 91 (1983) 49-60; G. M. Whitesides, J. E. Lilburn, R. P. Szajewski, Rates of Tjhioldisulfide Interchange Reactions Between Mono- and Ditiols and Ellman's Reagent, J. Org. Chem. 42 (1977) 332-338; P. Campbell, N. T. Nashed, Carboxypeptidase A Sataylzed Hydrolysis of Thiopeptide and Thioester Analogs of specific Substrates. An Effect on kcat for Peptide, But Not Ester, Substrates, J. Am. Chem Soc. 104 (1982) 5221-5226.) However, the reaction with free thiols has been shown to be much faster than that with thioacids. The reaction of DTNB with a free thiol can be complete within one minute at room temperature, as judged by the achievement of maximum absorbance at 412 nm. However, the reaction of DTNB with a thioacid may take up to 50-60 minutes at room temperature to achieve maximum absorbance at 412 nm, as already documented for the reaction of DTNB with thiobenzoic acid and with a peptidic thioacid. This likely explains the fact that DTNB has been primarily used to quantify free thiols via quantifying TNB from its absorbance at 412 nm.

However, in order to employ DTNB to quantify a thioacid released from an enzymatic reaction, it is essential to ensure a complete conversion of the thioacid to TNB in the presence of an excess amount of DTNB. Therefore, the inventors first performed a detailed kinetic analysis for the reaction between DTNB and the commercially available thioacetic acid in the absence of an enzyme and its substrate, as detailed by the inventors in D. G. Fatkins, Weiping Zheng, A Spectrophotometric Assay For Histone Deactylase 8, Anal. Biochem. 372 (2008) 82-88, incorporated herein by reference in its entirety. A₄₁₂ values were obtained and used to determine that maximum absorbance was achieved or closely approached starting at 60 minutes following DTNB addition for all the thioacetic acid concentrations examined. The A₄₁₂ values of TNB can be used to derive the amount of thioacetate present in the original assay mixture.

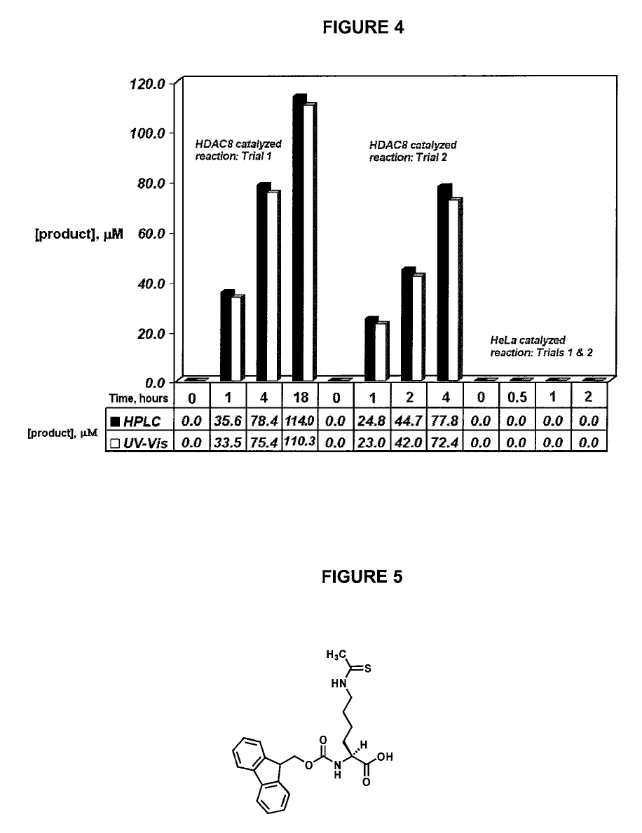

The HDAC8-catalyzed dethioacetylation of thioacetyl-lysine peptide is effectively monitored and quantified by using the current spectrophotometric assay as shown in FIG. 3. Using this spectrophotometric assay to quantify thioacetate in conjunction with an existing HPLC-based assay to quantify dethioacetylated peptide, identical reaction kinetics validating this spectrophotometric assay were achieved, as shown in FIG. 4. As shown in FIG. 4, thioacetyl-lysine peptide (see FIG. 3) did not serve as a substrate for the protein deacetylase enzymes, predominantly HDAC1 and HDAC2, present in the nuclear extract from the human HeLa cells. Furthermore, the same thioacetyl-lysin peptide did not serve as a substrate for the purified human HDAC1 and HDAC2. The foregong indicates that this spectrophotometric assay is a selective assay platform for determining the activity of HDAC8 when thioacetyl-lysine peptide is used.

Radioactive assay, HPLC assay, and fluorescent assay are the three currently available assay platforms for determining HDAC8 activity. As compared to these currently existing assays, the spectrophotometric assay according to the invention has several advantages. In one embodiment, the present invention provides the first HDAC8 spectrophotometric assay platform, and further provides the first HDAC8-selective assay platform. The prior art platforms specified hereinabove are responsive to the peptide product or the radio labeled acetate produced during the reaction between a substrate and the enzyme. However, the novel assay provided by the present invention is specific to the thioacetate product, not the peptide product, which can be further reacted to produce a product detectable by spectrophotometric analysis, thus rendering the assay novel and selective in terms of product measured and measurement mechanism. In addition, the assay platform according to the invention is environmentally friendly, efficient in terms of time and ease of use, cost-effective in that a readily available UV-Vis spectrophotometer is used to measure the spectrophotometric data, and, finally, the assay uses a substrate that is easily prepared through facile organic/peptide synthesis. The foregoing features of the novel spectrophotometric assay according to the invention allow the assay to find application for high-throughput screening of HDAC8-selective inhibitors and for selective reporting of HDAC8 activity under physiological and pathophysiological conditions. For example, the assay may be used in the screening process for newly developed cancer drugs, providing an efficient option to more cumbersome, time consuming, and costly prior art procedures.

With reference once again to FIG. 3, there is shown therein the peptide sequence that contains the novel ThAcK building block. This peptide sequence has the general formula

R¹NH—φₘ—(ThAcK)—φₙ—COR²

wherein R¹ is selected from the group consisting of H, acetyl (CH₃CO), and terbutyloxycarbonyl (^{t}Boc); R² is selected from the group consisting of hydroxyl (OH), amino (NH₂), and 7-amino-4-methylcoumarine (AMC); φ is a naturally occurring L-amino acid; ThAcK is L-N^{ε}-thioacetyl-lysine; m is 0-10; and n is 0-7 and when m and/or n is >2, φ may be the same amino acid or may be different amino acids. The naturally occurring amino acid, φ, may be any such amino acid, for example: alanine, arginine, asparagine, aspartic acid, glutamic acid, glutamine, glycine, histidine, isoleucine, leucine, lysine, methionine, phenylalanine, praline, serine, threonine, thryptophan, tyrosine, and valine.

With reference now to FIG. 5, the figure shows the structure of L-N^{α}-Fmoc-N^{ε}-thioacetyl-lysine. This compound is necessary for incorporating ThAcK, or L-N^{ε}-thioacetyl-lysine, into a peptide by using the Fmoc chemistry-based solid phase peptide synthesis (SPPS). The L-N^{α}-Fmoc-N^{ε}-thioacetyl-lysine is synthesized via the condensation of L-N^{α}-Fmoc-lysine with ethyl dithioacetate under slightly basic conditions. This Fmoc chemistry is known to those skilled in the art. Wellings, D. A., Atherton, E., Methods Enzymol. 1997, 289, 44.

In another embodiment, the present invention relates to a peptide substrate with at least two thioacetyl-lysines, the peptide substrate having the following generic formula:

R¹NH—SGRG(a)GG(b)GLG(c)GGA(d)RHR(e)VLR—COR²

wherein: R¹ is hydrogen (H), acetyl (CH₃CO), or tert-butyloxycarbonyl (^{t}Boc); R² is hydroxyl (OH), or amino (NH₂); (a) is L-lysine or L-N^{ε}-thioacetyl-lysine; (b) is L-lysine or L-N^{ε}-thioacetyl-lysine; (c) is L-lysine or L-N^{ε}-thioacetyl-lysine; (d) is L-lysine or L-N^{ε}-thioacetyl-lysine; and (e) is L-lysine or L-N^{ε}-thioacetyl-lysine. In this peptide substrate, at least two of (a)-(e) must be present as a thioacetyl-lysine.

In one embodiment, the present invention provides a spectrophotometric assay wherein purified HDAC8 is present. In this instance, the assay may be performed in vitro. The purified HDAC8 may be acquired from any known commercial source.

In another embodiment, the present invention provides a spectrophotometric assay wherein cell lysate containing a mixture of human enzymes, including the HDAC8 enzyme for which the assay platform is selective, is present. This use may be carried out in vitro or in vivo. The cell lysate may be acquired commercially. Optionally, the cell lysate may be generated in the laboratory from human cells, including healthy human cells and human cancer cells.

In still another embodiment, the present invention provides a spectrophotometric assay wherein the assay is perfromed inside a human cell containing the HDAC8 enzyme, necessarily an in vivo use. For this purpose, the peptide according to the invention must be able to penetrate the cell membrane either by itself or by its conjugate with another peptide.

Peptide synthesis and purification. The following peptides, used to generate the data shown in FIG. 4, were synthesized based on the Fmoc synthesis strategy referred to above on a commercial peptide synthesizer, such as that available from Protein Technologies Inc., Tucson, Ariz., USA: i) H₂N-KKGQSTSRHK(K)LMFKTEG-COOH (p53 peptide); and ii) H₂N-KKGQSTSRHK(ThAcK)LMFKTEG-COOH (thioacetyl-lysine p53 peptide).

For each peptide coupling reaction, 4 equivalents of a Fmoc-protected amino acid, 3.8-4.0 equivalents of the coupling reagent 2-(1H-benzotriazole-1-yl)-1,1,3,3-tetramethylaminium hexafluorophosphate (HBTU) and the additive N-hydroxybenzotriazole (HOBt) were used in the presence of 0.4 M 4-methylmorpholine/N,N-dimethylformamide (NMM/DMF) and the coupling reaction was allowed to proceed at room temperature for 1 hour. A 20% (v/v) piperidine/DMF solution was used for Fmoc removal. All the peptides were cleaved from the resins by reagent K (83.6% (v/v) trifluoroacetic acid, 5.9% (v/v) phenol, 4.2% (v/v) ddH₂O, 4.2% (v/v) thioanisole, 2.1% (v/v) ethanedithiol), precipitated in cold diethyl ether, and purified by reversed-phase high pressure liquid chromatography (RP-HPLC) on a preparative C18 column (100 Å, 2.14×25 cm). The column was eluted with a gradient of ddH₂O containing 0.05% (v/v) of trifluoroacetic acid and acetonitrile containing 0.05% (v/v) of trifluoroacetic acid at 10 mL/min and monitored at 214 nm. 214 nm is used to monitor or detect the peptide. The pooled HPLC fractions were stripped of acetonitrile and lyophilized to render all peptides as puffy white solids. Peptide purity (>95%) was verified by RP-HPLC on an analytical C18 column (100 Å, 0.46×25 cm). The column was eluted with a gradient of ddH₂O containing 0.05% (v/v) of trifluoroacetic acid and acetonitrile containing 0.05% (v/v) of trifluoroacetic acid at 1 mL/min and monitored at 214 nm. The molecular weights of all purified peptides were confirmed by either matrix assisted laser desorption ionization-time of flight (MALDI-TOF) or electrospray ionization (ESI) mass spectrometric analysis, generating the following data: p53 Peptide: MS (MALDI-TOF) m/e 2091 [M+H]⁺; thioacetyl-lysine p53 Peptide: MS (MALDI-TOF) m/e 2149 [M+H]⁺.

HPLC-based enzymatic assay. The HPLC data shown in FIG. 4 was generated using the following procedure. The HDAC8-catalyzed dethioacetylation reaction was performed in the same HDAC8 assay solution as above-described, except that no thioacetic acid was added but purified HDAC8 and its substrate (i.e. the above-described peptide ii) were added to final concentrations of 375 nM and 0.3 mM, respectively. An enzymatic reaction was initiated by the addition of HDAC8 at room temperature and was allowed to be incubated at room temperature until quenched at different time points with the following stop solution: 3.2 M guanidinium chloride in 100 mM of sodium phosphate (pH 6.8). One portion (40 uL) of the HDAC8 assay mixture was quenched with 80 uL of the above stop solution, and was analyzed as set forth above by RP-HPLC with a C18 analytical column (100 Å, 0.46×25 cm), eluting with the following gradient of double deionized water containing 0.05% (v/v) trifluoroacetic acid (mobile phase A) and acetonitrile containing 0.05% (v/v) trifluoroacetic acid (mobile phase B): linear increase from 0% B to 35% B from 0-40 min (1 mL/min), and ultraviolet (UV) monitoring at 214 nm. The enzymatically formed dethioacetylated product (i.e. the above-referenced peptide i) was confirmed by its comigration with the chemically synthesized authentic sample and by mass spectrometric analysis with MALDI-TOF, and was quantified by HPLC peak integration and comparison with that of the synthetic authentic sample. The same assay protocol was used when the HeLa nuclear extracts were used instead of human HDAC8. Nearly same assay protocols were used when purified HDAC1 and HDAC2 were employed instead of HDAC8, with the following exceptions: i) For HDAC1 assays, 50 mM Tris.HCl (pH 8.0) was used instead of 25 mM Tris.HCl (pH 8.0); ii) HDAC2 assays were performed at 30° C. instead of RT; iii) HDAC1 and HDAC2 assay solutions were all quenched with the following stop solution: 1.0 M HCl and 0.16 M acetic acid. These changes were instituted to more clearly assess the selectivity.

DTNB-based enzymatic assay. The DTNB data shown in FIG. 4 was generated using the following procedure. A second portion (300 UL) of the above HDAC8 assay mixture was quenched with 600 UL of the stop solution: 3.2 M guanidinium chloride in 100 mM of sodium phosphate (pH 6.8), and was analyzed by the DTNB-based assay procedure according to the invention, i.e. 60 min after the quench and DTNB (100 UL) addition, A₄₁₂ was recorded for each sample derived at different time points of the same assay reaction. Again, the same assay protocol was used when the HeLa nuclear extracts were used instead of human HDAC8. To measure the kinetic parameters for the thioacetyl-lysine p53 peptide as an in vitro HDAC8 substrate, all the assay reactions contained 375 nM of HDAC8, a range of substrate concentrations varied around the Kₘ value, and were allowed to proceed at room temperature for 1 hour before quenching. Reaction velocities were determined under initial condition (substrate turnover <10%). This experiment was performed twice, and each duplicate measurement agreed within 20%. By using a non-linear least squares approach with the computer program Kaleidagraph (Reading, Pa., USA), kinetic parameters (k_{cat} and Kₘ) were derived from the Michaelis-Menten plot based on the experimental data.

Assay with purified HDAC8. Using the thioacetyl-lysine p53 peptide as an in vitro substrate for HDAC8, the HPLC-based and the DTNB-based assays were both performed to evaluate the HDAC8-catalyzed dethioacetylation reaction, following the procedure detailed above. For the DTNB-based assay, the extinction coefficient (e) value for TNB reported in literature (13,700 M⁻¹ cm⁻¹) was used for our calculations even though a very close average value (13,505 M⁻¹ cm⁻¹) was also obtained from two standard curves (not shown) for thioacetic acid that were generated according to the DTNB-based assay procedure established according to the present invention. From the DTNB-based and the HPLC-based assay results shown in FIG. 4, it is clear that these two assay formats gave rise to mutually agreeable measurements (within 8%) for HDAC8 activity when the thioacetyl-lysine p53 peptide was employed as a substrate, even though the well-established HPLC-based assay measured the peptide product whereas the DTNB-based assay according to the present invention measured another product, i.e. thioacetic acid (or thioacetate under assay pH). The kinetic parameters for the in vitro HDAC8 substrate, i.e. the thioacetyl-lysine p53 peptide, were determined by the DTNB-based assay with k_{cat}=0.26±0.02 min⁻¹ and Kₘ=33.7±7.2 μM. From these kinetic parameters, it is clear that the DTNB-based assay according to the present invention offers another advantage in that reliable HDAC8 activity measurements can be made with low substrate concentrations, given that this Kₘ value is smaller than any of those for the currently available HDAC8 in vitro substrates.

Assay with HeLa nuclear extracts. In order to examine the selectivity of our newly developed DTNB-based spectrophotometric assay among different classical HDAC enzymes, we initially performed both the HPLC-based and the DTNB-based assays with the HeLa nuclear extracts enriched in HDAC1 and 2. The procedures for the HDAC8 assay were followed. It is apparent from FIG. 4 that no detectable dethioacetylation of the thioacetyl-lysine p53 peptide was observed with both the HPLC-based and the DTNB-based assay formats, when HDAC8 was replaced with the HeLa nuclear extracts. It was determined that because the intact thioacetyl-lysine p53 peptide was quantitatively recovered from the assay mixture as revealed by the HPLC-based assay (data not shown) there was no possibility that the degradation of the thioacetyl-lysine p53 peptide under assay conditions contributed to the lack of detectable dethioacetylation. However, under the same assay conditions, the corresponding acetyl-lysine p53 peptide was able to be deacetylated significantly by the HeLa nuclear extracts, in that 2.3% and 6.5% of substrate turnover were already observed at 30 min when 4 uL and 12 uL of the HeLa nuclear extracts (9 mg of protein per mL) were respectively used, with k_{obs}=0.23±0.01 uM/(min·4 uL of the nuclear extracts), as judged by HPLC analysis. This same phenomenon was also observed when AcNH—RH(AcK)—(AcK)—CONH₂ and AcNH—RH(ThAcK)(ThAcK)—CONH₂ were used, with the former being deacetylated significantly (k_{obs}=0.29±0.02 uM /(min·4 uL of the nuclear extracts) at 30 minutes), but no detectable dethioacetylation was observed for the latter. Again, intact AcNH—RH(ThAcK)(ThAcK)—CONH₂ was quantitatively recovered from the assay mixture as revealed by the HPLC-based assay (data not shown).

Assay with purified HDAC1 and HDAC2. Based on the above-described assay results with HeLa nuclear extracts, the spectrophotometric assay according to the present invention was shown to be selective for HDAC8 versus HDAC1 and 2 as well as other classical protein deacetylase enzymes present in the HeLa nuclear extracts, due to the incapability for HDACs other than HDAC8 to catalyze the dethioacetylation of the thioacetyl-lysine p53 peptide. Though FIG. 4 does not present this scenario, the data agrees with that presented fro the HeLa nuclear extracts. In order to provide further evidence for the selectivity of the assay format, analogous assays with HDAC1 and HDAC2 instead of the HeLa nuclear extracts were performed. Under the assay conditions detailed above, no detectable dethioacetylation of the thioacetyl-lysine p53 peptide was observed in both HDAC1 and HDAC2 assays, when the HPLC-based assay format was followed. However, under the same assay conditions, both HDAC1 and HDAC2 were shown to be able to deacetylate the corresponding acetyl-lysine p53 peptide with the following observed reaction rates: k_{obs}=0.41±0.23 min⁻¹ at 2 h for the former (HDAC1), and k_{obs}=6.78±2.39 min⁻¹ at 30 min for the latter (HDAC2). These assay results clearly support the conclusion that HDAC1 and 2 are unable to catalyze the dethioacetylation of the thioacetyl-lysine p53 peptide. In addition, the lack of detectable dethioacetylation of the thioacetyl-lysine p53 peptide in the assays with the HeLa nuclear extracts could not have been due to the possible interference from materials other than HDACs in the HeLa nuclear extracts with the recognition between HDACs and the thioacetyl-lysine p53 peptide.

Taken together, the assay results with both HeLa nuclear extracts and the purified HDAC1 and HDAC2 presented above demonstrate that the DTNB-based spectrophotometric assay according to the present invention is selective for HDAC8 versus HDAC1 and HDAC2 as well as other classical protein deacetylase enzymes present in the HeLa nuclear extracts. Though not wishing to be bound by any specific theory, it appears that while the catalytic domain is highly conserved among the classical HDAC enzymes, the acetyl-lysine binding pocket in HDAC8 is more malleable as compared to those in other classical HDACs. This could explain the observed capability and incapability respectively for HDAC8 and HDACs present in HeLa nuclear extracts to catalyze dethioacetylation reaction, due to the larger Van der Waals radius of S versus O.

Based on the foregoing, in one embodiment the present invention provides the first spectrophotometric HDAC8 assay via quantifying with DTNB the thioacetate product released from the enzymatic dethioacetylation of the thioacetyl-lysine peptide. Further, this novel assay is selective for HDAC8 versus HDAC1 and 2 as well as other classical HDAC enzymes. In addition to providing a fast and environment friendly assay platform, as compared to the currently existing HDAC8 assays, i.e. radioactive assay, HPLC assay, and fluorescence assay, the spectrophotometric assay according to the present invention also represents the most cost-effective format in that a readily available UV-Vis spectrophotomer is used and the substrate is readily obtained through facile organic/peptide synthesis. This novel spectrophotometric assay thus finds application for high-throughput screening of HDAC8-selective inhibitors and for selective reporting of HDAC8 activity under (patho)physiological conditions.

Although the invention has been described in detail with particular reference to certain embodiments presented herein, it has equal application to other embodiments wherein success can be anticipated. Variations, modifications and further application of the parameters and principles of the present invention will be obvious to those skilled in the art and the appended claims are intended to cover in the all such modifications and equivalents.

What we claim is:

1. A process to quantitatively measure the activity of human histone deactylase 8 (HDAC8), the process comprising:
a) providing a peptide substrate including at least one ThAcK amino acid;
b) providing HDAC8;
c) allowing the HDAC8 to react with the peptide reactant substrate to produce at least one peptide product and thioacetate; and
d) quenching HDAC8 activity;
e) adding DTNB to the quenched HDAC8;
f) allowing DTNB to react with thioacetate to produce TNB; and
g) quantitatively measuring the activity of HDAC8 by measuring the thioacetate concentration from the absorbance of TNB at 412 nm.

2. The process of claim 1 wherein the HDAC8 activity is quenched using a guanidinium chloride solution.

3. The process of claim 1 wherein the peptide substrate containing at least one ThAcK amino acid has the formula
R¹NH—φₘ—(ThAcK)—φₙ—COR²
wherein R¹ is selected from the group consisting of H, acetyl (CH₃CO), and terbutyloxycarbonyl (^{t}Boc); R² is selected from the group consisting of hydroxyl (OH), amino (NH₂), and 7-amino-4-methylcoumarine (AMC); φ is a naturally occurring L-amino acid; ThAcK is L-N^{ε}-thioacetyl-lysine; m is 0-10; and n is 0-7 and when m and/or n is >2, φ may be the same amino acid or may be different amino acids.

4. The process of claim 3 wherein φ is selected from alanine, arginine, asparagine, aspartic acid, glutamic acid, glutamine, glycine, histidine, isoleucine, leucine, lysine, methionine, phenylalanine, praline, serine, threonine, thryptophan, tyrosine, and valine.

5. The process of claim 1 wherein the HDAC8 is contained in a mixture of enzymes.

6. The process of claim 5 wherein the peptide substrate is selective for HDAC8.

7. The process of claim 1 wherein the HDAC8 is purified.

8. The process of claim 1 wherein the HDAC8 is present in human cells and the process is conducted in vitro.

9. The process of claim 1 wherein the HDAC8 is present in human cells and the process is conducted in vivo.

10. A selective peptide substrate comprising at least one ThAcK amino acid.

11. The selective peptide substrate of claim 10 containing at least one ThAcK amino acid has the formula
R¹NH—φₘ—(ThAcK)—φₙ—COR²
wherein R¹ is selected from the group consisting of H, acetyl (CH₃CO), and terbutyloxycarbonyl (^{t}Boc); R² is selected from the group consisting of hydroxyl (OH), amino (NH₂), and 7-amino-4-methylcoumarine (AMC); φ is a naturally occurring L-amino acid; ThAcK is L-N^{ε}-thioacetyl-lysine; m is 0-10; and n is 0-7.

12. The selectived peptide substrate of claim 11 wherein when m and/or n is >2, φ may be the same amino acid or may be different amino acids.

13. The selective peptide substrate of claim 11 wherein φ is selected from alanine, arginine, asparagine, aspartic acid, glutamic acid, glutamine, glycine, histidine, isoleucine, leucine, lysine, methionine, phenylalanine, praline, serine, threonine, thryptophan, tyrosine, and valine

14. A selective reagent for reporting HDAC8 activity comprising a peptide substrate containing at least one ThAcK amino acid, wherein the the substrate is a reagent for selectively reporting the activity of HDAC8 inside human cells.

15. The selective reagent of claim 14 wherein the peptide substrate comprises at least one ThAcK amino acid has the formula
R¹NH—φₘ—(ThAcK)—φₙ—COR²
wherein R¹ is selected from the group consisting of H, acetyl (CH₃CO), and terbutyloxycarbonyl (^{t}Boc); R² is selected from the group consisting of hydroxyl (OH), amino (NH₂), and 7-amino-4-methylcoumarine (AMC); φ is a naturally occurring L-amino acid; ThAcK is L-N^{ε}-thioacetyl-lysine; m is 0-10; and n is 0-7.

16. The selectived reagent of claim 15 wherein when m and/or n is >2, φ may be the same amino acid or may be different amino acids.

17. The selective reagent of claim 14 wherein the peptide substrate is selective for HDAC8 activity.

18. The selective reagent of claim 14 wherein the human cells are cancer cells.

19. A method for screening cancer-treating drugs comprising:
a) providing human cancer cells treated with a cancer-treating drug, wherein the cancer cells contain a mixture of human enzymes, one being HDAC8;
b) lysing the cells to render cell lysates;
c) providing a peptide substrate including at least one ThAcK amino acid having the general formula
R¹NH—φₘ—(ThAcK)—φₙ—COR²
wherein R¹ is selected from the group consisting of H, acetyl (CH₃CO), and terbutyloxycarbonyl (^{t}Boc); R² is selected from the group consisting of hydroxyl (OH), amino (NH₂), and 7-amino-4-methylcoumarine (AMC); φ is a naturally occurring L-amino acid; ThAcK is L-N^{ε}-thioacetyl-lysine; m is 0-10; and n is 0-7 and when m and/or n is >2, φ may be the same amino acid or may be different amino acids;
d) contacting the peptide substrate with the cancer cell lysates;
e) allowing the HDAC8 in the cancer cell lysates to react with the peptide substrate to produce at least one peptide product and thioacetate; and
f) quantitatively measuring the thioacetate concentration of the reaction of step (e) to determine the activity of HDAC8.

20. The method of claim 19, further including, between steps (e) and (f) the steps of: quenching HDAC8 activity; adding DTNB to the quenched HDAC8; and allowing the DTNB to react with the thioacetate to produce TNB.

21. The method of claim 20 wherein the quantitative measurement of step (f) is based on the activity of HDAC8 by measuring the thioacetate concentration from the absorbance of the TNB produced at 412 nm.

22. The method of claim 19 wherein the cells are lysed in vitro

23. The method of claim 19 wherein the cells are lysed in vivo.

24. The method of claim 19 wherein φ is selected from alanine, arginine, asparagine, aspartic acid, glutamic acid, glutamine, glycine, histidine, isoleucine, leucine, lysine, methionine, phenylalanine, praline, serine, threonine, thryptophan, tyrosine, and valine.

25. The process of claim 1 wherein the HDAC8 activity is quenched using a guanidinium chloride solution.
